(19) United States
(12) Patent Application Publication
(10) Pub. No.: US 2005/0207993 A1
(43) Pub. Date: Sep. 22, 2005

(54) Method and composition for breath freshening

(76) Inventors: Russel Bazemore, Naperville, IL (US); Daniel Zyck, North Riverside, IL (US); Lonnette Alexander, Chicago, IL (US); Vukasin Levakov, Chicago, IL (US); Jagriti Chander, Chicago, IL (US)

Correspondence Address: WRIGLEY & DREYFUS 28455;BRINKS HOFER GILSON & LIONE, P.O. BOX 10395, CHICAGO, IL, 60610, US

(21) Appl. No.: 10/989,905

(22) Filed: Nov. 15, 2004

Related U.S. Application Data
(60) Provisional application No. 60/481,637, filed on Nov. 13, 2003.

(51) Int. Cl. A61K 9/68; A61K 7/16; A61K 7/26

(52) U.S. Cl. 424/48; 424/58

(57) ABSTRACT

An oral composition for freshening the breath of consumers of tobacco products includes an oral cavity delivery agent, which can be a non-oxidizing oral cavity delivery agent, and an effective amount of a polyphenolic apple extract. The polyphenolic compounds in the apple extract react with nitrogen compounds in the comsumer's breath to reduce the concentration of the nitrogen compounds in the smoker's mouth. The oral products of this invention include chewing gums, edible thin film compositions, confectionary compositions, mouth sprays, and dentifrices. A method includes applying a non-oxidizing oral cavity delivery agent and a polyphenolic apple extract to the oral cavity of consumers of tobacco products.

### REFERENCE TO RELATED APPLICATION

The present application claims the benefit of the filing date under 35 U.S.C. § 119(e) of Provisional U.S. Patent Application Ser. No. 60/481,637, filed Nov. 13, 2003, which is hereby incorporated by reference in its entirety.

### TECHNICAL FIELD

The present invention relates, generally, to oral compositions having breath freshening properties and, more particularly, to a dentifrice, mouthwash, breath freshening capsule, chewing gum, confection, lozenge, mouth spray or edible film for freshening the breath of consumers of tobacco products, and to methods of freshening the breath of consumers of tobacco products.

### BACKGROUND

Breath freshening is a very important part of everyday life. In order to facilitate proper oral hygiene, oral cleansing and breath freshening practices should be conducted repeatedly throughout the day.

However, consumers of tobacco products typically smoke periodically throughout the day when oral cleansing and breath freshening may be difficult or inconvenient, depending on the nature of the breath freshening desired and the situation in which the breath freshening must occur. Brushing, flossing, cleaning your tongue and gargling using a variety of devices and compositions are common oral care practices well-suited for the privacy of one's home. But, such devices and compositions are less convenient to use away from the home where bathroom facilities might be scarce, unavailable or unsanitary.

It is known to incorporate active agents into oral products for the purpose of providing oral benefits including breath freshening. Such systems have the advantage of providing a rapid effect and convenient delivery. Consumers of tobacco products sometimes use mints, chewing gums or cough drops to freshen breath, but these products have only limited effectiveness in combating tobacco breath.

U.S. Pat. No. 6,294,161 discloses breath freshening formulations against tobacco that include a natural extract that contains polyphenolic compounds, mixed with enzymes. The enzymes are taught to be necessary to oxidize the polyphenols to quinones to activate them. Testing of these compounds in development of the present invention, however, has shown these quinones to be ineffective against tobacco breath.

### BRIEF SUMMARY

It has surprisingly been found that polyphenolic compounds can be included in chewing gum and other oral compositions without the need for oxidation and still provide breath freshening capabilities effective against tobacco related bad breath. Hence, the present invention uses polyphenolic compounds, such as apple extracts, in a non-oxidizing oral cavity delivery composition.

The present invention relates to the composition of, and methods of using an oral product. Specifically, the present invention relates to oral products intended for breath freshening properties, formulated to freshen breath of consumers of tobacco products. More specifically, the present invention relates to a dentifrice, mouthwash, breath freshening capsule, chewing gum, confection, lozenge, mouth spray, or edible film containing an effective amount of a polyphenolic compound, by which the inventive composition effectively freshens breath of consumers of tobacco products, through the consumption of the dentifrice, chewing gum, confection, lozenge, mouths spray, toothpaste or edible film product.

In one form, the present invention is a chewing gum or any variation including but not limited to bubble gums, pellets, gum balls or sticks. Chewing gums may be coated or not coated, and be of a variety of flavors, shapes and sizes.

In another form, the invention is a confectionery composition including but not limited to hard candy, chewing candy, filled candy and pressed tablets.

In yet another form, the invention is an edible thin film.

In still another form, the invention is a dentifrice.

In one aspect, the invention includes an oral composition for freshening the breath of consumers of tobacco products. The oral composition includes a non-oxidizing oral cavity delivery agent, and an effective amount of a polyphenolic apple extract.

In another aspect, the invention includes a chewing gum composition for freshening the breath of consumers of tobacco products. The composition includes a water soluble bulk portion, flavoring agents, a gum base portion, and a polyphenolic compound. The polyphenolic compound reacts with nitrogen compounds in the consumer's breath to reduce the concentration of the nitrogen compounds.

In yet another aspect, the invention includes an edible film composition for freshening the breath of consumers of tobacco products. The composition includes a film forming agent and a polyphenolic compound.

In still another aspect, the invention includes a confectionary composition for freshening the breath of consumers of tobacco products. The confectionary composition includes a polyphenolic compound and at least one of a sugar or sugar alcohol. The polyphenolic compound reacts with nitrogen compounds in the consumer's breath to reduce the concentration of the nitrogen compounds.

In a further aspect, the invention includes a breath spray composition for freshening the breath of consumers of tobacco products. The composition includes an aqueous flavor solution and a polyphenolic compound. The polyphenolic compound reacts with nitrogen compounds in the consumer's breath to reduce the concentration of the nitrogen compounds.

In a still further aspect, the invention includes a method of binding pyridine in the mouth of an individual after smoking. The method includes consuming an oral composition containing a pyridine binding agent, where the pyridine binding agent is present in an amount sufficient to bind pyridines to reduce malodor in the mouth of said individual.

### BRIEF DESCRIPTION OF THE DRAWING

FIG. 1 is a plot of cigar aftertaste intensity versus time in sensory panelists following chewing no gum, a control gum, peppermint Eclipse®, and a gum formulated with 4% Applephenon®;

FIG. 2 is a plot of cigarette aftertaste intensity versus time in sensory panelists following chewing no gum, a control gum, a gum with 2% Applephenon, and control gum with 4% added Applephenon®;

FIG. 3 is a histogram showing the effect of chewing no gum, a control gum, and a gum with added 4% Applephenon® on odorous compounds extracted from the oral cavity following smoking a cigar; and

FIG. 4 is a histogram showing the effect of adding 1% (wt/wt) Applephenon® to a standard solution containing 100 ppm pyridines in ethanol and water.

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

It is known to use chewing gum, confections and thin films as a vehicle for delivering components to the oral cavity which provide oral benefits such as breath freshening properties. Such systems have the advantage of providing a consumer with a convenient and inexpensive method for maintaining fresh breath throughout the course of the day. The present invention relates to a method and composition which reduces the odor of the breath of an individual after smoking by adding a polyphenolic compound. Polyphenolic compounds are extracts taken from items such as apples, grapes, blueberries, fruits, vegetables and the like. The method of reducing odor from the breath of an individual after smoking involves the individual placing a product which contains a polyphenolic compound in his or her mouth. Such products may be chewing gums, confections, edible films, mouthwashes, mouth sprays, breath freshening capsules, dentifrices, and the like. The compositions of such products are also disclosed herein.

More specifically, the preferred embodiments of the present invention are oral compositions containing polyphenolic apple extract, which freshens breath of consumers after consuming a cigar or cigarette. The malodorous compounds from tobacco smoke are known as pyridines (ethyl pyridine, 2,3,-dimethyl pyridine). Polyphenolic apple extract is an extract obtained from unripe apples. A preferred polyphenolic apple extract is Applephenon®, which may be obtained from A.M. Todd, Kalamazoo, Mich. Polyphenolic apple extract contains polyphenols and has a slight apple flavor. Polyphenolic apple extract reduces the malodor associated with the pyridines and pyrazines of tobacco breath.

Sensory experiments were conducted with cigar smokers and cigarette smokers. Sensory readings were taken at timepoints immediately after smoking, 30 minutes after smoking, 1 hour after smoking and 2 hours following smoking. Self perception and second party breath odor judges evaluated individuals that had no treatment after smoking for cigar only, individuals who chewed a control chewing gum with no actives, individuals who chewed Eclipse®, which is a Wm. Wrigley Jr. Company breath freshening product, or a gum that had the same formulation as the control gum, but with Applephenon® added.

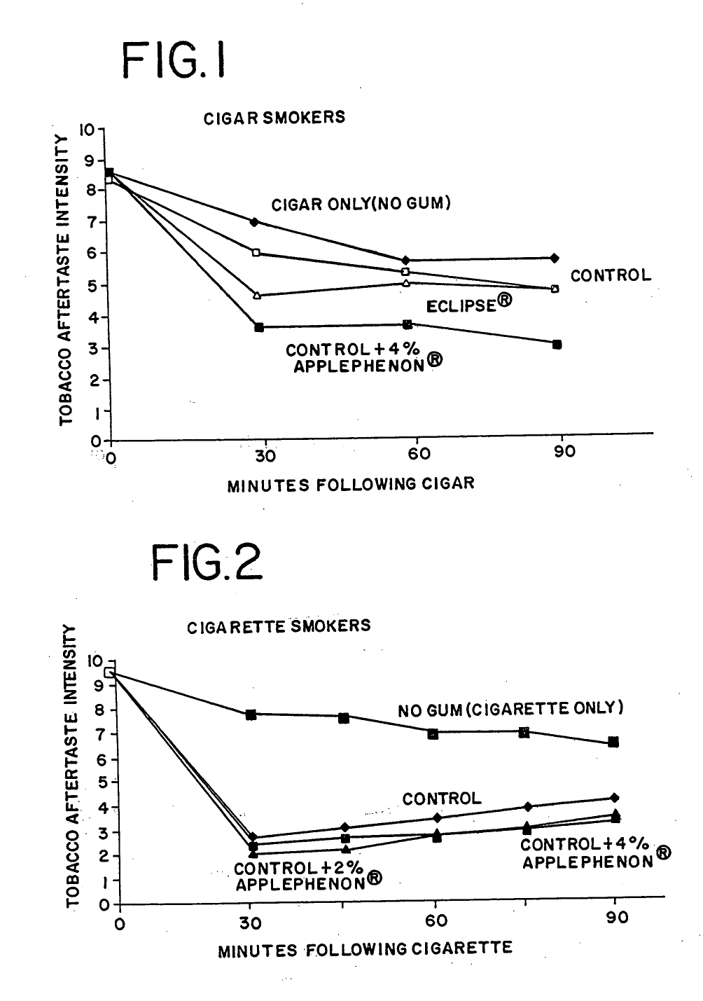

Results from recently conducted sensory testing with cigarette smokers indicate a trend almost identical to that exhibited by previous internal testing with cigar smokers, namely a gum formulated with Applephenon®, a polyphenolic substance extracted from immature apples, significantly decreases tobacco aftertaste intensity vs. a control with no added polyphenolic apple extract as measured over time (FIG. 1 and FIG. 2).

With cigars, aftertaste intensity was reduced the greatest, and remained the lowest following chewing gum formulated with 4% (wt/wt) Applephenon®. Aftertaste intensity scores were significantly lower than those obtained following chewing the control gum for each time period (P<0.05). Additionally aftertaste intensity scores obtained following chewing the gum with 4% Applephenon® (based on uncoated weight) were significantly lower than scores measured after chewing Eclipse® Winterfresh™ gum, a Wm. Wrigley Jr. Company chewing gum with proven breath freshening effectiveness, at 60 min. and 90 min time intervals. Lastly, chewing gum, any gum, significantly reduced cigar aftertaste intensity vs. chewing no gum at all for each time period.

With cigarettes, aftertaste intensity determined after chewing a gum formulated with 2% Applephenon® (based on uncoated weight) was significantly different from the control gum with no actives at each time beyond 30 minutes (P<0.1). With gum formulated with 4% Applephenon®, intensities were significantly different from the control for 60 min. and 75 min. time intervals (P<0.1). There were no significant differences in aftertaste intensities between gums formulated with 2% and 4% Applephenon®. As with cigars, chewing any gum significantly reduced aftertaste intensity below that of chewing no gum at all for each time period.

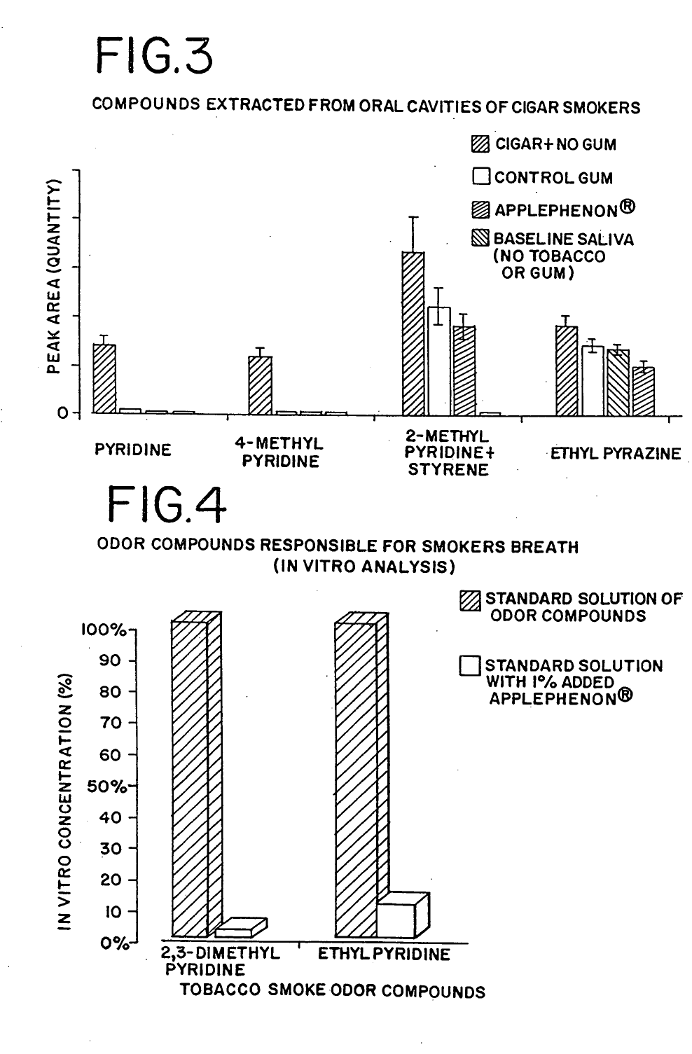

Analytical results corroborate sensory findings. One of the most aroma active groups of compounds found to contribute to the odor of tobacco breath (determined by gas chromatography-olfactometry coupled with SPME extraction), pyridines, are almost entirely eliminated by chewing a gum to which has been added a polyphenolic apple extract (FIG. 3). Pyrazines and pyrroles, two additional classes of related aromatic nitrogenous compounds, are also reduced, but at a lower level.

The aromatic nitrogenous component reduction effect was further investigated to ascertain the value of polyphenolic apple extract treatment vs. normal salivary action induced by chewing any gum (from FIG. 3 differences in trace pyridine levels extracted from oral cavities of cigar smokers and Applephenon® treated gums were negligible). Headspace samples from solutions that contained 100 ppm pyridines with and without 1% Applephenon® were measured. Results clearly showed polyphenolic apple extract efficacy in reducing pyridine headspace levels (FIG. 4).

In summary, sensory analyses with cigars and cigarettes, as well as analytical analyses of in-vitro and in-vivo odor compounds responsible for tobacco smoke odor, indicate the polyphenol blend in polyphenolic apple extract reduces the concentration of compounds responsible for tobacco breath odor.

It has further been discovered that maintaining the pH of the oral cavity during use of the product so that it is less than the pKa of pyridine improves the effectiveness of the polyphenolic apple extract in reducing the odor-causing compounds in smoker's breath. The pKa of pyridine in the mouth of a consumer of tobacco products is about 4.5. It is believed that a pH level in this range or below improves the binding effectiveness of the polyphenolic compounds. Accordingly, in one formulation of the invention, an oral cavity delivery agent includes a food grade acid so that it creates a pH of less than about 4.5 in the oral cavity when the product is initially placed in the oral cavity.

In one form of the present invention, a chewing gum composition contains about 0.1 wt. % to about 6 wt. % polyphenolic apple extract, by weight of the chewing gum composition (based on uncoated weight).

In another form, a chewing gum composition contains about 1.5 wt. % to about 4.5 wt. % polyphenolic apple extract by weight of the chewing gum composition (based on uncoated weight).

To create a desired pH level in the smoker's mouth, the chewing gum composition preferably includes a food grade acid, such as citric acid, malic acid, tartaric acid, fumaric acid, and the like. The chewing gum composition preferably includes about 0.5 wt. % to about 8.0 wt. % food grade acid. In a more preferred formulation, the food grade acid is present in an amount of about 2 wt. % about 6 wt. % of the product. And, in a most preferred formulation, the food grade acid is present in an amount of about 3 wt. % to about 5 wt. % of the product.

The chewing gum composition can further include food acceptable salts of zinc or copper, such as zinc and copper salts of gluconic acid, lactic acid, acetic acid, citric acid and combinations thereof. The chewing gum composition can further include a cooling agent or a heating agent, or both. The chewing gum composition further comprises a phosphate selected from the group consisting of pyrophosphate or polyphosphate.

The chewing gum composition can include a tooth whitening agent or an an anti-plaque agent, or both.

There are several methods which may be used to enhance the release of the polyphenolic apple extract extract from the chewing gum. The polyphenolic apple extract may be encapsulated, spray dried, formulated into a coating on a chewing gum product, or present in a liquid center and combinations thereof.

In general, a chewing gum composition typically comprises a water soluble portion, flavoring agents and a gum base portion. The water soluble portion dissipates with a portion of the flavoring agent over a period of time during chewing. The gum base portion is retained in the mouth throughout the chew.

The insoluble gum base generally comprises elastomers, resins, fats and oils, softeners and inorganic fillers. The gum base may or may not include wax. The insoluble gum base can constitute approximately 5% to about 95% by weight of the chewing gum, more commonly the gum base comprises 10% to about 50% of the gum. In another preferred formulation, the gum base comprises approximately 25% to about 35% by weight, of the chewing gum.

In a particular formulation, the chewing gum base of the present invention contains about 20% to about 60% by weight synthetic elastomer, up to about 30% by weight natural elastomer, about 5% to about 55% by weight elastomer plasticizer, about 4% to about 35% by weight filler, about 5% to about 35% by weight softener, and optional minor amounts (about 1% or less by weight) of miscellaneous ingredients such as colorants, antioxidants, etc.

Synthetic elastomers may include, but are not limited to, polyisobutylene with GPC weight average molecular weight of about 10,000 to about 95,000, isobutylene-isoprene copolymer (butyl elastomer), styrene copolymers having styrene-butadiene ratios of about 1:3 to about 3:1, polyvinyl acetate having GPC weight average molecular weight of about 2,000 to about 90,000, polyisoprene, polyethylene, vinyl acetate vinyl laurate copolymer having vinyl laurate content of about 5% to about 50% by weight of the copolymer, and combinations thereof.

Preferred ranges for polyisobutylene are 50,000 to 80,000 GPC weight average molecular weight and for styrene are 1:1 to 1:3 bound styrene for polyvinyl acetate are 10,000 to 65,000 GPC weight average molecular weight with the higher molecular weight polyvinyl acetates typically used in bubble gum base, and for vinyl acetatelaurate, vinyl laurate content of 10%.

Natural elastomers may include natural rubber, such as smoked or liquid latex and guayule, as well as natural gums such as jelutong, lechi caspi, perillo, sorva, massaranduba balata, massaranduba chocolate, nispero, rosindinha, chicle, gutta hang kang, and combinations thereof. The preferred synthetic elastomer and natural elastomer concentrations vary depending on whether the chewing gum in which the base is used is adhesive or conventional, or a bubble gum or regular gum, as discussed below. Preferred natural elastomers include jelutong, chicle, sorva and massaranduba balata.

Elastomer plasticizers may include, but are not limited to, natural rosin esters such as glycerol esters or partially hydrogenated rosin, glycerol esters of polymerized rosin, glycerol esters of partially dimerized rosin, glycerol esters of rosin, pentaerythritol esters of partially hydrogenated rosin, methyl and partially hydrogenated methyl esters of rosin, pentaerythritol esters of rosin; synthetics such as terpene resins derived from alpha beta and/or any suitable combinations of the foregoing. The preferred elastomer plasticizers will also vary depending on the specific application, and on the type of elastomer which is used.

Fillers/texturizers may include magnesium and calcium carbonate, ground limestone, silicate types such as magnesium and aluminum silicate, clay, alumina, talc, titanium oxide, mono-, di- and tri-phosphate, cellulose polymers, such as wood, and combinations thereof.

Softeners/emulsifiers may include tallow, hydrogenated tallow, hydrogenated and partially hydrogenated vegetable oils, cocoa butter, glycerol monostearate, glycerol triacetate, lecithin, mono and triglycerides, acetylated monoglycerides, fatty acids (e.g. stearic, palmitic, oleic and linoleic acids), and combinations thereof.

Colorants and whiteners may include FD&C dyes and lakes, fruit and vegetable extracts, titanium dioxide, and combinations thereof.

The base may or may not include wax. An example of a wax free gum base is disclosed in U.S. Pat. No. 5,286,500, the disclosure of which is incorporated herein by reference.

In addition to a water insoluble gum base portion, a typical chewing gum composition includes a water soluble bulk portion and one or more flavoring agents. The water soluble portion can include bulk sweeteners, high-intensity sweeteners, flavoring agents, softeners, emulsifiers, colors, acidulants, fillers, antioxidants, and other components that provide desired attributes.

Softeners are added to the chewing gum in order to optimize the chewability and mouthfeel of the gum. The softeners, which are also known as plasticizers and plasticizing agents, generally constitute between approximately 0.5% to about 15% by weight of the chewing gum. The softeners may include glycerin, lecithin, and combinations thereof. Aqueous sweetener solutions such as those containing sorbitol, hydrogenated starch hydrolysates, corn syrup and combinations thereof, may also be used as softeners and binding agents in chewing gum.

Bulk sweeteners include both sugar and sugarless components. Bulk sweeteners typically constitute about 5% to about 95% by weight of the chewing gum, more typically, about 20% to about 80% by weight, and more commonly, about 30% to about 60% by weight of the gum. Sugar sweeteners generally include saccharide components commonly known in the chewing gum art, including but not limited to, sucrose, dextrose, maltose, dextrin, dried invert sugar, fructose, levulose, galactose, corn syrup solids, and the like, alone or in combination. Sugarless sweeteners include, but are not limited to, sugar alcohols such as sorbitol, mannitol, xylitol, hydrogenated starch hydrolysates, maltitol, and the like, alone or in combination.

High-intensity artificial sweeteners can also be used, alone or in combination, with the above. Preferred sweeteners include, but are not limited to, sucralose, aspartame, NAPM derivatives such as neotame, salts of acesulfame, altitame, saccharin and its salts, cyclamic acid and its salts, glycyrrhizinate, dihydrochalcones, thaumatin, monellin, and the like, alone or in combination. In order to provide longer lasting sweetness and flavor perception, it may be desirable to encapsulate or otherwise control the release of at least a portion of the artificial sweetener. Such techniques as wet granulation, wax granulation, spray drying, spray chilling, fluid bed coating, coacervation, and fiber extension may be used to achieve the desired release characteristics.

Combinations of sugar and/or sugarless sweeteners may be used in chewing gum. Additionally, the softener may also provide additional sweetness such as with aqueous sugar or alditol solutions.

If a low calorie gum is desired, a low caloric bulking agent can be used. Examples of low caloric bulking agents include: polydextrose; Raftilose, Raftilin; Fructooligosaccharides (NutraFlora); palatinose oligosaccharide; guar gum hydrolysate (Sun Fiber); or indigestible dextrin (Fibersol). However, other low calorie bulking agents can be used.

A variety of flavoring agents can also be used, if desired. The flavor can be used in amounts of about 0.1 to about 15 weight percent of the gum, and preferably, about 0.2% to about 5% by weight. Flavoring agents may include essential oils, synthetic flavors or mixtures thereof including, but not limited to, oils derived from plants and fruits such as citrus oils, fruit essences, peppermint oil, spearmint oil, other mint oils, clove oil, oil of wintergreen, anise and the like. Artificial flavoring agents and components may also be used. Natural and artificial flavoring agents may be combined in any sensorially acceptable fashion.

Flavoring may include a cooling agent to enhance the flavor and perceived breath freshening of the product. Cooling agents include menthol, ethyl p-menthane carboxamide, N-ethyl-p-menthane-3-carboxamide (WS-3), N,2,3-trimethyl-2-isopropyl-butanamide (WS-23), menthyl glutarate FEMA 4006, menthyl succinate, menthol PG carbonate, menthol EG carbonate, menthyl lactate, menthone glyceryl ketal, menthol glyceryl ether, N-tertbutyl-p-menthane-3-carboxamide, p-menthane-3-carboxylic acid glycerol ester, methyl-2-isopryl-bicyclo (2.2.1) heptane-2-carboxamide, menthol methyl ether, menthol glyceryl ether, 3,3,5-trimethylcyclohexanol (Homomenthol), isopulegol, and combinations thereof.

In addition, to the active ingredients of the present invention, additional active ingredients or medicaments may be added for various purposes. If the medicament or active ingredient is water soluble in the chewing gum, it preferably will include a base/emulsifier system which leads to the desired concentration of the medicament in the saliva (more hydrophilic balance). If the medicament or active is water insoluble, the chewing gum preferably includes a base/emulsifier system which leads to the desired concentration of the medicament in the saliva (more lipophilic balance).

The present invention may also optionally contain a heating agent. The heating agent, in small amounts, potentiates the effect of the cooling agent. The heating agent may also substitute for the cooling agent in the present invention if the experience of heat, tingling or itching is desired. The heating agents of the present invention are capsicum oleoresin, capsaicin, piperine, gingerol, shoagol, cinnamic aldehyde, ginger oleoresin, cinnamon oleoresin, cassia oleoresin, black pepper oleoresin, pepper oleoresin and combinations thereof.

In manufacturing the chewing gum including the active agent or ingredient, the active agent or medicament is added. The smaller the amount of active ingredient used, the more necessary it becomes to preblend that particular ingredient to assure uniform distribution throughout the batch of gum. Whether a preblend is used or not, the active agent or medicament should be added within the first five minutes of mixing. For faster release, the active agent may be added late in the process.

Optionally, the chewing gum of the present invention may include additional breath freshening, anti-microbial or oral health ingredients. Food acceptable metallic salts selected from zinc and copper salts of gluconic acid, zinc and copper salts of lactic acid, zinc and copper salts of acetic acid, zinc and copper salts of citric acid and combinations thereof. Zinc lactate and copper gluconate may be added to broaden the breath freshening capacity of this product to reduce odors from etiology other than smoking or tobacco products. A chewing gum of the present invention preferably contains a phenolic compound further comprising zinc lactate and/or copper gluconate. These salts may be used in ranges of about 0.1% to about 1.0% by weight of the chewing gum composition.

Anti-microbial essential oils and flavor components such as peppermint, methyl salicylate, thymol, menthol, MBE, eucalyptol, cinnamic aldehyde, and combinations thereof, may also be incorporated into products of the present invention.

Dental health ingredients such as fluoride salts, phosphate salts, proteolytic enzymes, lipids, anti-microbials, calcium, electrolytes, protein additives, dental abrasives and combinations thereof, may also be incorporated into products of the present invention.

In general, chewing gum is manufactured by sequentially adding the various chewing gum ingredients to a commercially available mixer known in the art. After the ingredients have been thoroughly mixed, the gum mass is discharged from the mixer and shaped into the desired form such as rolling sheets and cutting into sticks, extruding into chunks or casting into pellets, which are then coated or panned.

Generally, the ingredients are mixed by first melting the gum base and adding it to the running mixer. The base may also be melted in the mixer itself. Color or emulsifiers may also be added at this time. A softener such as glycerin may also be added at this time, along with syrup and a portion of the bulking agent. Further portions of the bulking agent are added to the mixer. Flavoring agents are typically added with the final portion of the bulking agent. Other optional ingredients are added to the batch in a typical fashion, well known to those of ordinary skill in the art.

The entire mixing procedure typically takes from five to fifteen minutes, but longer mixing times may sometimes be required. Those skilled in the art will recognize that many variations of the above described procedure may be followed.

Chewing gum base and chewing gum product have been manufactured conventionally using separate mixers, different mixing technologies and, often, at different factories. One reason for this is that the optimum conditions for manufacturing gum base, and for manufacturing chewing gum from gum base and other ingredients such as sweeteners and flavors, are so different that it has been impractical to integrate both tasks. Chewing gum base manufacturing, on the one hand, involves the dispersive (often high shear) mixing of difficult-to-blend ingredients, such as elastomers, fillers, elastomer plasticizers, base softeners/emulsifiers and sometimes wax, and typically requires long mixing times. Chewing gum product manufacture, on the other hand, involves combining the gum base with more delicate ingredients such as product softeners, bulk sweeteners, high intensity sweeteners and flavoring agents using distributive (generally lower shear) mixing, for shorter periods. However, it is also known to make gum base and chewing gum in the same mixer, as taught in U.S. Pat. No. 5,800,847, which is hereby incorporated by reference. All such methods of making chewing gum can be used in practicing the present invention.

The following are examples of formulations of polyphenolic apple extract in chewing gum. The examples are not intended to exclude other variations in formulations, and the present invention is not limited to these formulations. Table 1 illustrates exemplary chewing gum formulations including food grade acids for creating a desired pH level in a smoker's mouth.

TABLE 1
Chewing Gum Formulas (% weight)

| Ingredient | Example 1 | Example 2 |
|---|---|---|
| Base | 31.196 | 31.196 |
| Sorbitol | 45.485 | 47.485 |
| Glycerin | 5.163 | 5.163 |
| Applephenon | 4.000 | 4.000 |
| Fruit Flavor | 2.700 | 2.700 |
| Talc | 2.455 | 2.455 |
| Citric Acid | 1.500 | 1.000 |
| Malic Acid | 1.500 | 1.000 |
| Tartaric Acid | 1.500 | 1.000 |
| Fumaric Acid | 1.500 | 1.000 |
| Encapsulated APM | 1.872 | 1.872 |
| Cooling agents | 0.600 | 0.600 |
| Lecithin | 0.259 | 0.259 |
| Acesulfame K | 0.218 | 0.218 |
| Water | 0.052 | 0.052 |
| Total % | 100.00 | 100.00 |

Table 2 illustrates additional formulations of polyphenolic apple extract in chewing gum.

TABLE 2
Chewing Gum Formulas (% weight)

| Ingredient | Example 3 | Example 4 | Example 5 | Example 6 | Example 7 |
|---|---|---|---|---|---|
| Gum Base | 25.21 | 25.21 | 25.21 | 25.21 | 25.21 |
| Lecithin | 0.17 | 0.17 | 0.17 | 0.17 | 0.17 |
| NaHCO₃ | 0.25 | 0.25 | 0.25 | 0.25 | 0.25 |
| Sorbitol | 50.86 | 49.86 | 47.56 | 45.26 | 49.81 |
| Polyphenolic apple extract | — | 1.00 | 3.00 | 5.00 | 0.50 |
| Mannitol | 4.25 | 4.25 | 4.25 | 4.25 | 4.25 |
| Lycasin/Glycerin | 8.51 | 8.51 | 8.51 | 8.51 | 8.51 |
| Glycerin | 8.50 | 8.50 | 8.50 | 8.50 | 8.50 |
| Encapsulated | 0.67 | 0.67 | 0.67 | 0.67 | 0.67 |
| Sweetener | | | | | |
| Flavor | 1.58 | 1.58 | 1.58 | 1.58 | 1.58 |
| Zinc Lactate | — | — | 0.30 | — | 0.25 |
| Cu Gluconate | — | — | — | 0.60 | 0.30 |
| Total Percent | 100.00 | 100.00 | 100.00 | 100.00 | 100.00 |

In one form of the present invention, an effective amount of polyphenolic compound for breath freshening of smokers breath is present in an edible film formulation. The amount of polyphenolic compound is preferably present in an amount up to about 40% by weight of the edible film formulation. The polyphenolic compound is preferably a polyphenolic apple extract and is present in an amount that up to about 8% of the weight of the edible film product and, more preferably, up to about 5% by weight of the edible film product.

The present invention provides edible film formulations for oral mucoadhesion and methods of using and making same. In particular, the edible films of the present invention can include one or more of several film forming agents, such as maltodextrins, fillers (e.g., microcrystalline cellulose (MCC)) and hydrocolloids (e.g., sodium aliginate), and Pullulan, which can be effectively utilized to prepare stand alone edible films. In this regard, the edible films can provide a physiologically acceptable film, which is suitably adapted to adhere to oral surfaces of an oral cavity and rapidly dissolve therein.

The edible films of the present invention can be utilized to deliver or release oral care agent(s). Such agents include, anti-microbial agents and salivary stimulants to treat, for example, halitosis, dental plaque, gingivitis, xerostomia, dry mouth, like oral conditions or combinations thereof. Further, the oral care edible film can act as a breath freshener effective against malodor.

The oral cleansing and breath freshening effects of the edible film of the present invention can be achieved by entrapping the oral care and breath freshening agents within the oral cavity to provide extended efficacy. In this regard, the highly dissolvable edible film can act as a medium through which a pharmaceutically active oral agent can be administered via a mucous membrane of the oral cavity.

Further, the edible films can include a variety of other suitable ingredients, such as softeners, colorants, flavoring agents, emulsifiers, surfactants, thickening agents, binding agents, sweeteners, fragrances, other like ingredients or combinations thereof.

The edible films preferably include a mixture of at least three types of film forming agents, such as Pullulan, maltodextrins, fillers and hydrocolloids. It should be appreciated that the preferred edible film can be composed of one or more different compounds associated with the film forming agents.

The maltodextrin component preferably constitutes between about 5% to about 60% by dry weight of the edible film, preferably about 20% to about 40% by dry weight. The maltodextrin component can be processed in any suitable way.

The hydrocolloid can provide thickness and decrease brittleness of the edible films. The hydrocolloid can include any suitable type, amount and number of hydrocolloids. The hydrocolloid can constitute between about 10% to about 50% by dry weight of the edible film, preferably about 20% to about 30% by dry weight. The hydrocolloid can be derived from, for example, natural seaweeds, natural seed gum, natural plant exudates, natural fiber extracts, biosynthetic gums, gelatins, biosynthetic process starch or cellulosic materials, alginates, sodium alginate, calcium alginate, carrageenans, guar gum, locust gum, tara gum, gum arabic, ghatti gum, agar gum, xanthan gum, pectin, other like hydrocolloid source material or combinations thereof.

Any suitable food-grade bulk filler can also be added to the edible film. This can reduce any slimy texture as well as provide structure to the film, thereby making it more palatable. The filler can constitute about 5% to about 30% by dry weight of the film, preferably about 15% to about 25% by dry weight. The filler can include, for example, microcrystalline cellulose, cellulose polymers, such as wood, magnesium and calcium carbonate, ground limestone, silicates, such as magnesium and aluminum silicate, clay, talc, titanium dioxide, mono-calcium phosphate, di-calcium phosphate, tri-calcium phosphate, other like bulk fillers or combinations thereof.

As previously discussed, a variety of other suitable ingredients can be added to the edible film of the present invention. For example, any suitable active ingredient or medicament for oral cleansing, breath freshening or the like can be added to the film formulation. The ingredients can include, for example, a pH control agent, such as urea and buffers, inorganic components for tartar or caries control, such as phosphates and fluorides, an additional breath freshening agent, such as zinc gluconate, an anti-plaque/anti-gingivitis agent, such as cholorhexidene, CPC, and triclosan, a saliva stimulating agent including, for example, food acids such as citric, lactic, maleic, succinic, ascorbic, adipic, fumaric and tartaric acids, a pharmaceutical agent, a nutraceutical agent, a vitamin, a mineral, other like ingredients or combinations thereof. Zinc lactate and copper gluconate may be added to broaden the breath freshening capacity of this product to reduce odors from etiology other than smoking or tobacco products. The edible film can also contain a phenolic compound further comprising zinc lactate and/or copper gluconate.

The medicaments can be delivered or released into the oral cavity for effective oral treatment, such as oral cleansing and/or breath freshening. In this regard, the film forming agent of the edible film can act to entrap the medicaments within the oral cavity, thereby providing extended efficacy thereof. In doing so, it is believed that the edible film compositions of the present invention more uniformly release the medicament into the oral cavity for absorption via open wounds or mucous membrane in a greater manner than could be previously achieved. Moreover, it is also believed that the mixture of film-forming agents of the present invention can entrap the medicament within the oral cavity for an extended period of time to prolong and enhance the effects of the medicament.

If reduced levels of film forming agents are utilized, softeners can be used to reduce the brittleness of the resulting films. The softeners, which are also known as plasticizers or plasticizing agents, generally constitute up to about 20% by dry weight of the film, preferably about 2% to about 10% by dry weight. The softeners can also include plasticizers containing, for example, sorbitol and other polyols, glycerin, polyethylene glycol, propylene glycol, hydrogenated starch hydrolysates, corn syrups, other like material or combinations thereof.

The edible film formulations of the present invention can also include colorants or coloring agents, which can be used in any suitable amount to produce the desired color. Coloring agents can include, for example, natural food colors and dyes suitable for food, drug and cosmetic applications. The colorants are typically knows as FD&C dyes and lakes.

A variety of flavoring agents can also be added to the edible films. Any suitable amount and type of artificial and/or natural flavoring agents can be used in any sensorially acceptable fashion. For example, the flavor can constitute about 0.1% to about 20% by dry weight of the film, preferably about 10% to about 15%. The flavoring agents can include, for example, essential oils, synthetic flavors or mixtures, including but not limited to, oils delivered from plants and fruits such as citrus oils, fruit essences, peppermint oil, spearmint oil, other mint oils, clove oils, oil of wintergreen, anise and the like, flavor oils with germ killing properties such as menthol, eucalyptol, thymol, like flavoring agents or combinations thereof.

In addition to the flavoring agents described above, cooling agents, such as those described above for the chewing gum formulation may be included in amounts of about 0.01% to about 2% by weight of the edible film formulation.

The flavor can be enhanced and evenly distributed throughout the product by emulsification. Any suitable amount and type of natural and/or synthetic food-grade emulsifier can be used. For example, the emulsifier can include lecithin, food-grade non-ionic emulsifiers, such as fatty acids (C₁₀-C₁₈), mono and diacyl glycerides, ox bile extract, polyglycerol esters, polyethylene sorbitan esters, propolyene glycol, sorbitan monopalmitate, sorbitan monosterate, sorbitan tristerate, enzyme modified lecithin, hyroxylated lecithins, other like emulsifiers or combinations thereof.

The flavors can be emulsified by any suitable emulsification process, such as mechanical processing, vigorous stirring, intense pressure fluctuations that occur in turbulent flow such as homogenization, sonication, colloid milling and the like.

In addition to flavoring agents, the edible film can also include heating agents, such as those described above for the chewing gum formulation. Generally, the heating agent may be present in amounts of about 0.01% to about 3% by weight of the edible film formulation.

The present invention provides methods of producing the edible film formulations. In general, the edible film formulations are prepared by forming a base solution that includes one or more film forming agents, such as maltodextrins, hydrocolloids and fillers, and processing the base solution to form an edible film. Typically, the base solution is prepared by adding an initial mixture of dry ingredients to water that is stirred.

To the base solution, additional ingredients, such as flavor/emulsifier blends, sweeteners, softeners, color, the like or combinations thereof, can be added. Preferably, the solution is stirred continuously and heated at a temperature ranging from about 40° C. to about 60° C. The solution then can be dried in any suitable manner, thereby, forming the edible film.

It should be appreciated that any suitable type, number and arrangement of process procedures or steps (i.e. mixing, heating, drying, cooling, addition of ingredients), process parameters (i.e. temperature, pressure, pH, process times) or the like can be utilized.

By way of example and not limitation, the following examples illustrate various formulations of the edible film of the present invention.

TABLE 3
Edible Film Formulations (% weight)

| Ingredient | Example 8 | Example 9 | Example 10 | Example 11 | Example 12 |
|---|---|---|---|---|---|
| Maltodextrin | 25.05 | 46.80 | 31.20 | 36.80 | 21.00 |
| Sodium Alginate | 22.50 | — | 19.00 | — | 12.00 |
| Calcium Alginate | — | 15.15 | — | 11.15 | — |
| Carrageenan | — | — | — | — | 12.00 |
| Microcrystalline | 25.75 | 9.00 | 18.30 | 13.00 | 20.00 |
| Cellulose | | | | | |
| Calcium Carbonate | — | 2.45 | — | — | — |
| Glycerin | 12.25 | 10.00 | 8.00 | — | 9.5 |
| Sorbitol | — | — | — | 6.00 | 1.55 |
| Propylene Glycol | — | — | 3.65 | 5.00 | — |
| Menthol | 1.00 | 0.05 | — | 1.25 | — |
| Eucalyptol | — | 0.05 | — | 1.00 | — |
| Maleic Acid | — | — | — | — | 1.35 |
| Citric Acid | — | — | 1.25 | — | 1.00 |
| Chlorohexidene | 1.85 | — | — | 1.00 | — |
| Triclosan | — | 1.25 | — | 1.00 | — |
| Flavor | 9.40 | 11.00 | 12.00 | 14.00 | 10.00 |
| High Intensity | 1.25 | 1.00 | 1.05 | 1.45 | 1.50 |
| Sweetener | | | | | |
| Polyphenolic apple | 1.00 | 3.00 | 5.00 | 8.00 | 10.00 |
| extract | | | | | |
| Color | 0.05 | 0.05 | 0.05 | 0.05 | 0.10 |
| Zinc Lactate | — | 0.20 | — | 0.10 | — |
| Copper Gluconate | — | — | 0.50 | 0.20 | — |
| Total % | 100.00 | 100.00 | 100.00 | 100.00 | 100.00 |

TABLE 4
Edible Film Formulations (% weight)

| | | | | Example | Example |
|---|---|---|---|---|---|
| Ingredient | Example 13 | Example 14 | Example 15 | 16 | 17 |
| Maltodextrin | 35.00 | 30.35 | 20.15 | 25.00 | 10.00 |
| Sodium Alginate | 22.15 | 19.10 | 10.00 | 28.15 | — |
| Carrageenan | — | — | | — | 20.15 |
| Microcrystalline | 20.00 | 18.00 | 17.00 | 17.00 | 18.00 |
| Cellulose | | | | | |
| Gum Arabic | — | — | 11.00 | — | — |
| Glycerin | 7.30 | 15.00 | 7.30 | 7.30 | 7.30 |
| Flavor | 11.00 | 11.00 | 11.00 | 11.00 | 11.00 |
| Lecithin | 2.00 | 2.00 | 2.00 | 2.00 | 2.00 |
| High Intensity | 1.50 | 1.50 | 1.50 | 1.50 | 1.50 |
| Sweetener | | | | | |
| Polyphenolic apple | 1.00 | 3.00 | 20.00 | 8.00 | 30.0 |
| extract | | | | | |
| Color | 0.05 | 0.05 | 0.05 | 0.05 | 0.05 |
| Total % | 100.00 | 100.00 | 100.00 | 100.00 | 100.00 |

In yet another form of the present invention, an effective amount of polyphenolic apple extract is present for novel flavor experience in a confectionery formulation. In one preferred formulation, the polyphenolic apple extract is present in an amount up to 10% by weight of the confectionery product. In another preferred formulation, the polyphenolic apple extract is present in an amount of about 4% by weight of the confectionery product. In yet another preferred formulation, the polyphenolic apple extract is present in an amount of about 2% to about 6% by weight of the confectionery product.

Confectionery products for this invention may be hard candies, chewy candies, coated chewy center candies, gummy candies, coated gummy candies and tabletted candies. By way of example, the hard candy is primarily comprised of corn syrup and sugar, and derives its name from the fact that it contains only 1.0% to 4% moisture. In appearance, these types of candies are solid, but they are actually supercooled liquids, which are far below their melting points. There are different types of hard candies. Glass types are usually clear or made opaque with dyes; and grained types, which are always opaque.

The continuous making process of the deposited glass types, with a sugar base are as follows. Sugar corn syrup mixture is spread over a cylinder heated by high pressure steam. Rapid heat exchange causes the water in the syrup to evaporate. The cooked syrup is discharged, and colors and flavors are added. These can be conveyed directly to hoppers which then discharge directly into molds.

The candy is conveyed to batch rollers, which shapes and sizes the batch. The candy enters a former, which shapes the individual pieces into discs, balls, barrels. The composition of the present invention can be made into any shape: circles, squares, triangles, animal shapes, or any other novelty molding available. The candy is then cooled, wrapped and packaged.

Chewy candy is made similar to hard candy, but also contains fat, such as coconut oil, palm kernal oil, soybean oil and the like. Gelatin, gum Arabic and carrageenan gums may also be added.

Gummy candy is cooked at about 240 degrees and deposited at about 25% moisture into molds. The bulk ingredients are sugar, corn syrup or polyols with gelatin at about 2-10%. The polyol, sugar or corn syrup solids and/or maltodextrins are compressed with high pressure (1000 psi to 15000 psi) to form. Gummy candy also contains lubricants such as magnesium stearate, calcium stearate, fumed silicon dioxide and the like. Examples of polyols used in the present invention are sorbitol, xylitol, maltitol and the like.

For grained types of candy, water and sugar, or corn syrup, are the basic components being mixed with other ingredients, and cooked at high temperatures (290° F. to 310° F.), causing the water to turn to steam. The product is transferred to a cooling wheel, where it is collected in about 150 pound batches, placed in a pulling machine to aerate the product, and the flavor is added. The candy is transferred to batch rollers where it is shaped and sized. The candy then enters a former, which shapes the individual pieces. The candy is cooled at a relative humidity of 35% and enters a rotating drum where it is coated with a fine sugar. The candy is then conveyed to the graining room for four hours at 90° F. and 60% humidity. The entrapped air and moisture causes the product to grain.

The present invention can be of a variety of shapes, flavors and sizes. The present invention may contain sugar or may be sugarless.

Flavors used in confectionary products of the present invention may be peppermint oils, citrus oils, arvensis, fruit flavors, spearmint oils and the like.

Colors used in confectionary products the present invention are colorants are typically known as FD&C dyes and lakes.

By way of example and not limitation, the following examples illustrate various formulation of the confectionery composition of the present invention.

TABLE 5
Novel Flavor Candy Formulations (% by weight)

| | Example | Example | Example | Example | Example |
|---|---|---|---|---|---|
| Ingredient | 18 | 19 | 20 | 21 | 22 |
| Corn Syrup | 44.51 | 43.25 | — | — | 48.00 |
| Sugar | 53.49 | 50.00 | — | — | 46.90 |
| Sorbitol | — | — | 93.80 | 95.77 | — |
| Polyphenolic | 0.75 | 1.92 | 4.2 | 0.48 | 3.85 |
| apple | | | | | |
| extract | | | | | |
| Color | 0.49 | 1.00 | 0.60 | 0.80 | 0.50 |
| Zinc Lactate | — | 1.92 | — | 0.65 | 0.45 |
| High- | — | — | 0.20 | 0.20 | — |
| Intensity | | | | | |
| Sweetener | | | | | |
| Peppermint | 0.08 | 1.91 | — | 2.10 | 0.90 |
| Heating | 0.01 | — | — | — | 0.03 |
| Agent | | | | | |
| Copper | 0.67 | — | 1.20 | — | — |
| Gluconate | | | | | |
| Total % | 100.00 | 100.00 | 100.00 | 100.00 | 100.00 |

In yet another aspect of the invention, the oral cavity delivery agent is a breath spray. Preferred breath sprays are aqueous solutions or emulsions with a flavoring agent, and can be formulated with or without an ethanol co-solvent.

The breath spray composition preferably includes about 0.2 wt. % to about 6 wt. % polyphenolic apple extract. In a more preferred formulation, the polyphenolic apple extract is present in an amount of about 0.5 wt. % about 3 wt. % polyphenolic apple extract. And, in a most preferred formulation, the polyphenolic apple extract is present in an amount of about 1 wt. % to about 2 wt %. polyphenolic apple extract.

Preferably, sweeteners are also added to the aqueous solution. The breath spray can also include a cooling agent, such as described above. A preferred cooling agent is N-2,3-trimehyl-2-isoproply-butamide available, for example, under the tradename W-23 from Millenium Chemicals Inc., Hunt Valley, Md. The solution is delivered to the consumer in a pressurized or pump spray container, which allows the consumer to spray the solution into the oral cavity.

By way of example and not limitation, the following example illustrates one breath spray formulation of the present invention.

TABLE 6
Breath Spray Formulation (% by weight)

| Ingredient | Example 23 |
|---|---|
| menthol | 1.96 |
| WS-23 | 0.37 |
| Sucralose | 0.29 |
| Peppermint flavor | 0.98 |
| Ethanol | 72.69 |
| Applephenon ® | 0.98 |
| Water | 22.73 |
| Total % | 100.00 |

The components listed above in Table 6 are preferably added together in a pump spray bottle having about a 10 mL capacity.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

1. An oral composition for freshening the breath of consumers of tobacco products, the oral composition comprising:
(a) a non-oxidizing oral cavity delivery agent; and
(b) an effective amount of a polyphenolic apple extract.

2. The composition of claim 1 wherein the non-oxidizing oral cavity delivery agent comprises a chewing gum composition and the polyphenolic apple extract comprises about 0.1 wt. % to about 6 wt. % of the chewing gum composition.

3. The composition of claim 2 wherein the polyphenolic apple extract comprises about 1.5 wt. % to about 4.5 wt. % of the chewing gum composition.

4. The composition of claim 1 wherein the non-oxidizing oral cavity delivery agent comprises an edible film composition and the polyphenolic apple extract comprises up to about 40 wt. % of the edible film composition.

5. The composition of claim 4 wherein the polyphenolic apple extract comprises up to about 8 wt. % of the edible film composition.

6. The composition of claim 4 wherein the polyphenolic apple extract comprises up to about 5 wt. % of the edible film composition.

7. The composition of claim 1 wherein the non-oxidizing oral cavity delivery agent comprises a confectionary composition and the polyphenolic apple extract comprises up to about 10 wt. % of the confectionary composition.

8. The composition of claim 7 wherein the polyphenolic apple extract comprises up to about 4 wt. % of the confectionary composition.

9. The composition of claim 1 wherein the non-oxidizing oral cavity delivery agent comprises a dentifrice.

10. The chewing gum composition of claim 1 wherein non-oxidizing oral cavity delivery agent creates a pH of less than about 4.5 in the consumer's mouth.

11. A chewing gum composition for freshening the breath of consumers of tobacco products, the composition comprising:
(a) a water soluable bulk portion;
(b) at least one flavoring agent;
(c) a gum base portion; and
(d) a polyphenolic compound,
wherein the polyphenolic compound reacts with nitrogen compounds in the comsumer's breath to reduce the concentration of the nitrogen compounds.

12. The chewing gum composition of claim 11 wherein the polyphenolic compound comprises polyphenolic apple extract comprising about 0.1 wt. % to about 6 wt. % of the chewing gum composition.

13. The chewing gum composition of claim 11 wherein the polyphenolic compound comprises polyphenolic apple extract comprising about 1.5 wt. % to about 4.5 wt. % of the chewing gum composition.

14. The chewing gum composition of claim 11 further comprising an anti-plaque agent.

15. The chewing gum composition of claim 11 further comprising a cooling agent wherein the cooling agent comprises at least one of menthol, ethyl p-menthane carboxamide, N-2,3-trimethyl-2-isopryl-butanamide, menthyl glutarate FEMA 4006, menthyl succinate, menthol PG carbonate, menthol EG carbonate, menthyl lactate, menthone glyceryl ketal, menthol glyceryl ether, N-tertbutyl-p-menthane-3-carboxamide, p-menthane-3-carboxylic acid glycerol ester, methyl-2-isopryl-bicyclo(2.2.1) heptane-2-carboxamide, menthol methyl ether, and combinations thereof.

16. The chewing gum composition of claim 11 further comprising a phosphate selected from the group consisting of pyrophosphate or polyphosphate.

17. The chewing gum composition of claim 111 wherein the water soluable bulk portion comprises at least one of bulk sweeteners, high-intensity sweeteners, flavoring agents, softeners, emulsifiers, colors, acidulants, fillers, and antioxidants.

18. The chewing gum composition of claim 11 wherein the water soluable bulk portion comprises one or more bulk sweeteners comprising about 30 wt. % to about 60 wt. % of the chewing gum composition.

19. The chewing gum composition of claim 11 wherein the at least one flavoring agent comprises at least one of essential oils and synthetic flavors.

20. The chewing gum composition of claim 11 wherein the chewing gum base portion comprises about 20 wt. % to about 60 wt. % synthetic elastomer, up to about 30 wt. % natural elastomer, about 5 wt. % to about 55 wt. % elastomer plasticizer, about 4 wt. % to about 35 wt. % filler, and about 5 wt. % to about 35 wt. % softener.

21. The chewing gum composition of claim 20 wherein the synthetic elastomer comprises at least one of polyisobutylene with GPC weight average molecular weight of about 10,000 to about 95,000; isobutylene-isoprene copolymer (butyl elastomer); styrene copolymers having styrene-butadiene ratios of about 1:3 to about 3:1; polyvinyl acetate having GPC weight average molecular weight of about 2,000 to about 90,000; polyisoprene; polyethylene; and a vinyl acetate/vinyl laurate copolymer having vinyl laurate content of about 5 wt. % to about 50 wt. % of the copolymer.

22. The chewing gum composition of claim 20 wherein the elastomer plasticizers comprises at least one of natural rosin esters, glycerol esters of polymerized rosin, glycerol esters of partially dimerized rosin, glycerol esters of rosin, pentaerythritol esters of partially hydrogenated rosin, methyl and partially hydrogenated methyl esters of rosin, pentaerythritol esters of rosin, and terpene resins.

23. The chewing gum composition of claim 11 further comprising a food acceptable metallic salt and an anti-microbial agent.

24. The chewing gum composition of claim 11 further comprising at least one of zinc lactate and copper gluconate.

25. The chewing gum composition of claim 11 further comprising a heating agent.

26. The chewing gum composition of claim 11 further comprising a tooth whitening agent.

27. The chewing gum composition of claim 11 wherein the composition creates a pH of less than about 4.5 in the consumer's mouth.

28. An edible film composition for freshening the breath of consumers of tobacco products, the composition comprising:
(a) a film forming agent; and
(b) a polyphenolic compound.

29. The edible film composition of claim 28 wherein the polyphenolic compound comprises polyphenolic apple extract comprising up to about 40 wt. % of the edible film composition.

30. The edible film composition of claim 28 wherein the polyphenolic compound comprises polyphenolic apple extract comprising up to about 8 wt. % of the edible film composition.

31. The edible film composition of claim 28 wherein the polyphenolic compound comprises polyphenolic apple extract comprising up to about 5 wt. % of the edible film composition.

32. The edible film composition of claim 28 wherein the film forming agent comprises at least one of a maltodextrine, a filler, a hydrocolloid, and Pullulan.

33. The edible film composition of claim 28 wherein the the film forming agent comprises maltodextrine at a level of about 5 wt. % to about 60 wt. % of the edible film.

34. The edible film composition of claim 28 wherein the film forming agent comprises maltodextrine at a level of about 20 wt. % to about 40 wt. % of the edible film.

35. The edible film composition of claim 28 wherein the film forming agent comprises hydrocolloid at a level of about 10 wt. % to about 50 wt. % of the edible film.

36. The edible film composition of claim 28 wherein the film forming agent comprises hydrocolloid at a level of about 20 wt. % to about 30 wt. % of the edible film.

37. The edible film composition of claim 35 wherein the hydrocolloid comprises at least one of natural seaweeds, natural seed gum, natural plant exudates, natural fiber extracts, biosynthetic gums, gelatins, biosynthetic process starch, cellulosic materials, alginates, sodium alginate, calcium alginate, carrageenans, guar gum, locust gum, tara gum, gum arabic, ghatti gum, agar gum, xanthan gum, and pectin.

38. The edible film composition of claim 28 wherein the film forming agent comprises a filler at a level of about 5 wt. % to about 30 wt. % of the edible film.

39. The edible film composition of claim 32 wherein the film forming agent comprises filler at a level of about 15 wt. % to about 25 wt. % of the edible film.

40. The edible film composition of claim 38 wherein the filler comprises at least one of microcrystalline cellulose, cellulose polymers, magnesium carbonate, calcium carbonate, ground limestone, magnesium silicate, aluminum silicate, clay, talc, titanium dioxide, mono-calcium phosphate, di-calcium phosphate, and tri-calcium phosphate.

41. The edible film composition of claim 28 further comprising a softening agent.

42. The edible film composition of claim 41 wherein the softening agent comprises up to about 20 wt. % of the edible film.

43. The edible film composition of claim 41 wherein the softening agent comprises about 2 wt. % to about 10 wt. % of the edible film.

44. The edible film composition of claim 41 wherein the softening agent comprises at least one of sorbitol, glycerin, polyethylene glycol, propylene glycol, hydrogenated starch hydrolysates, and corn syrups.

45. The edible film composition of claim 41 further comprising at least one of zinc lactate and copper gluconate.

46. The edible film composition of claim 28 further comprising a coloring agent.

47. The edible film composition of claim 46 wherein the coloring agent comprises at least one of a natural food color, an FD&C dye, and an FD&C lake.

48. The edible film composition of claim 28 further comprising a flavoring agent.

49. The edible film composition of claim 48 wherein the flavoring agent comprises about 0.1 wt. % to about 20 wt. % of the edible film.

50. The edible film composition of claim 48 wherein the flavoring agent comprises about 10 wt. % to about 15 wt. % of the edible film.

51. The edible film composition of claim 48 wherein the flavoring agent comprises at least one of synthetic flavors, citrus oils, fruit essences, peppermint oil, spearmint oil, other mint oils, clove oils, oil of wintergreen, anise menthol, eucalyptol, thymol.

52. The edible film composition of claim 48 further comprising an emulsifier.

53. The edible film composition of claim 52 wherein the emulsifier comprises at least one of lecithin, fatty acids (C₁₀-C₁₈), mono and diacyl glycerides, ox bile extract, polyglycerol esters, polyethylene sorbitan esters, propolyene glycol, sorbitan monopalmitate, sorbitan monosterate, sorbitan tristerate, enzyme modified lecithin, and hyroxylated lecithins.

54. A confectionary composition for freshening the breath of consumers of tobacco products, the composition comprising:
(a) at least one of a sugar or a sugar alcohol; and
(b) a polyphenolic compound,
wherein the polyphenolic compound reacts with nitrogen compounds in the comsumer's breath to reduce the concentration of the nitrogen compounds.

55. The confectionary composition of claim 54 wherein the polyphenolic compound comprises polyphenolic apple extract comprising up to about 10 wt. % of the confectionary formulation.

56. The confectionary composition of claim 54 wherein the polyphenolic compound comprises polyphenolic apple extract comprising about 2 wt. % to about 6 wt. % of the confectionary formulation.

57. The confectionary composition of claim 54 wherein the polyphenolic compound comprises polyphenolic apple extract comprising up to about 4 wt. % of the confectionary formulation.

58. The confectionary composition of claim 54 wherein the composition comprises one of hard candies, chewy candies, coated chewy center candies, gummy candies, coated gummy candies, and tabletted candies.

59. The confectionary composition of claim 54 wherein the composition comprises a chewy candy, wherein the chewy candy further comprises at least one of coconut oil, palm kernal oil, soybean oil.

60. The confectionary composition of claim 54 wherein the composition comprises a chewy candy, wherein the chewy candy further comprises at least one of gelatin, gum Arabic, and carrageenan.

61. The confectionary composition of claim 54 wherein the composition comprises a candy, and wherein the gummy candy further comprises gelatin comprising about 2 wt. % to about 10 wt. % of the confectionary formulation.

62. The confectionary composition of claim 54 wherein the composition comprises a tabletted candy, and the tabletted candy further comprises at least one of magnesium stearate, calcium stearate, and fumed silicon dioxide.

63. The confectionary composition of claim 62 wherein the tabletted candy further comprises at least one of xylitol, maltitol, and sorbitol.

64. The confectionary composition of claim 54 wherein the composition comprises a grained candy, wherein the grained candy further comprises entrapped air and moisture.

65. The confectionary composition of claim 54 wherein the sugar comprises corn syrup.

66. The confectionary composition of claim 54 wherein the sugar alcohol comprises sorbitol.

67. A method of freshening the breath of a consumer of tobacco products by applying an oral composition to the oral cavity of the consumer wherein the oral composition comprises a non-oxidizing oral cavity delivery agent and a polyphenolic apple extract.

68. The method of claim 67 wherein applying a non-oxidizing oral composition comprises applying a chewing gum composition, wherein the polyphenolic apple extract comprises about 0.1 wt. % to about 6 wt. % of the chewing gum composition.

69. The method of claim 68 wherein the polyphenolic apple extract comprises about 1.5 wt. % to about 4.5 wt. % of the chewing gum composition.

70. The method of claim 67 wherein applying a non-oxidizing oral cavity delivery agent comprises applying an edible film composition, wherein the polyphenolic apple extract comprises up to about 40 wt. % of the edible film composition.

71. The method of claim 70 wherein the polyphenolic apple extract comprises up to about 8 wt. % of the edible film composition.

72. The method of claim 70 wherein the polyphenolic apple extract comprises up to about 5 wt. % of the edible film composition.

73. The method of claim 67 wherein applying a non-oxidizing oral cavity delivery agent comprises applying a confectionary composition, wherein the polyphenolic apple extract comprises up to about 10 wt. % of the confectionary composition.

74. The method of claim 73 wherein the polyphenolic apple extract comprises up to about 4 wt. % of the confectionary composition.

75. The method of claim 67 wherein applying a non-oxidizing oral cavity delivery agent comprises applying a dentifrice.

76. A breath spray composition for freshening the breath of consumers of tobacco products, the composition comprising:
(a) an aqueous flavor solution; and
(b) a polyphenolic compound,
wherein the polyphenolic compound reacts with nitrogen compounds in the comsumer's breath to reduce the concentration of the nitrogen compounds.

77. The breath spray composition of claim 76 further comprising ethanol.

78. The breath spray composition of claim 76 further comprising a sweetener.

79. The breath spray composition of claim 76 further comprising a cooling agent.

80. The breath spray composition of claim 76 wherein the polyphenolic compound comprises a polyphenolic apple extract comprising about 0.8 to about 1.2 wt. % of the breath spray composition.

81. A method of binding pyridine in the mouth of an individual after smoking, which method comprises:
(a) consuming an oral composition containing a pyridine binding agent;
(b) wherein said pyridine binding agent is present in an amount sufficient to bind pyridines to reduce malodor in the mouth of said individual.

82. The method of claim 81 wherein consuming an oral composition containing a pyridine binding agent comprises consuming an oral composition containing polyphenolic apple extract.
